(19) United States
(12) Patent Application Publication
(10) Pub. No.: US 2014/0107602 A1
(43) Pub. Date: Apr. 17, 2014

(54) My Best Friend Forever Panty

(71) Applicant: Jane Hamilton, Torrance, CA (US)

(72) Inventor: Jane Hamilton, Torrance, CA (US)

(21) Appl. No.: 13/651,382

(22) Filed: Oct. 13, 2012

(51) Int. Cl. A61F 13/84

(52) U.S. Cl. CPC A61F 13/8405
USPC 604/359

(57) ABSTRACT

Today's women have been patiently waiting for competent monthly menstrual protection. My Best Friend for Forever Panty are garments that will save most women from embarrassing moments, support their body shape and give them a slimmer look, while displaying a feminine design. My Best Friend Forever Panty is a Disposable Sanitary Garment that offer the following solutions for menstrual needs: Three Designs to choose from: Bikini Panties, Girl Shorts and Slimming Shapers. This will give our buyers a choice to choose the garment that best fits their body. My Best Friend Forever Panty provides protection for various stages of menstrual flow—from light to heavy. The three layer pad is thick enough to absorb excessive fluid and provides anti-leakage protection product that acts as a barrier between clothing to prevent soiling). This product is ideal for women who suffer from symptoms derived from fibroid tumors such as heavy bleeding and clotting. Odor neutralizing infused in the crouch to prevent unpleasant odor from the effects of being on the menstrual cycle.

### PROCESS/MAKING: CONSTRUCTION AND USE OF INVENTION

This product consist of four entities:
1. Body (Outer Layer)—Provides full coverage, absorbency, and dryness over the lower abdomen area to prevent leakage of menstrual flow. Also provides all over slimming, shaping, smoothing and contouring to the tummy, waist, hips, thigh, and rear areas of the body.
2. Core lining (moisture barrier)—provides maximum coverage, absorbency, anti-leakage, and odor control (saturated with homeopathic odor resistant scent) throughout the inner lining as well as in the crotch area of the panty.
3. Inner Material (material closest to the skin)—thin soft non-woven-perforated material (for comfort and dryness to skin, which allows fluid to flow to the cotton area).
4. Overlay material (fusing material)—made of nylon, spandex, latex, type material. This material is cut to the specifications of the (bikini panty, boy shorts or slimming shapers) and it also assists in fusing and sealing the following materials: Body (outer layer), Core Lining (moisture barrier) and Inner Material (material closest to the skin) creating one strong entity. Assemble Process: Fuse Body (outer layer), Core Lining (coverage material) and inner material together. Cut into desired pattern (bikini panties, boy shorts. Slimming Shapers). Then seal all three layers together with a seamless, nylon, spandex latex, type material with minimal hem. Place a disposable, optional print design material overlay on the front, sides and back of panty creating an attractive, print design.

### BRIEF DETAILED DESCRIPTION

My Best Friend Forever Bikini Panty (Invention by Jane Hamilton)

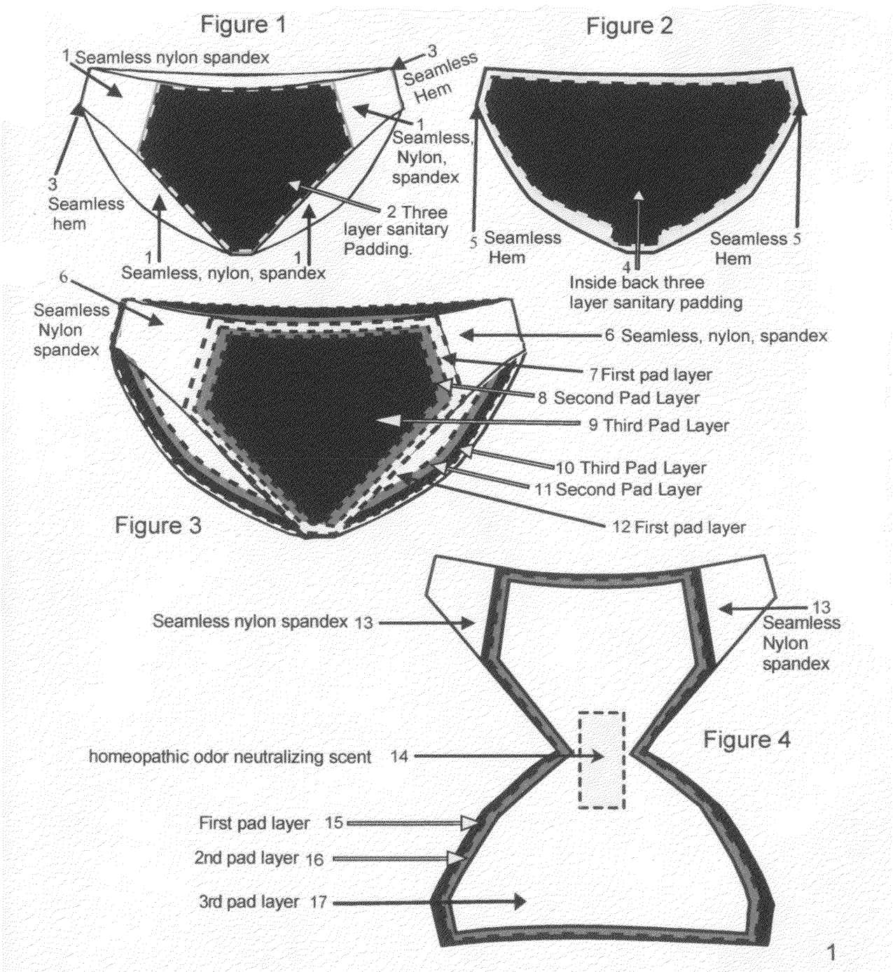

FIG. 1 Front View:
1. Seamless Nylon Spandex
2. Three Layer Sanitary Padding
3. Seamless Hem

FIG. 2 Back View:
4. Inside back Three layer sanitary padding
5. Seamless Hem

FIG. 3 Front View Layered:
6. Seamless Nylon Spandex
7. First Pad Layer (front of Panty)
8. Second Pad Layer (front of Panty)
9. Third Pad Layer (front of Panty)
10. Third Pad Layer (Back of Panty)
11. Second Pad Layer(Back of Panty)
12. First Pad Layer (Back of Panty)

FIG. 4 (Inside View):
13. Seamless Nylon Spandex
14. Homeopathic Order Neutralizing Scent
15. First pad Layer
16. Second Pad Layer
17. Third Pad Layer

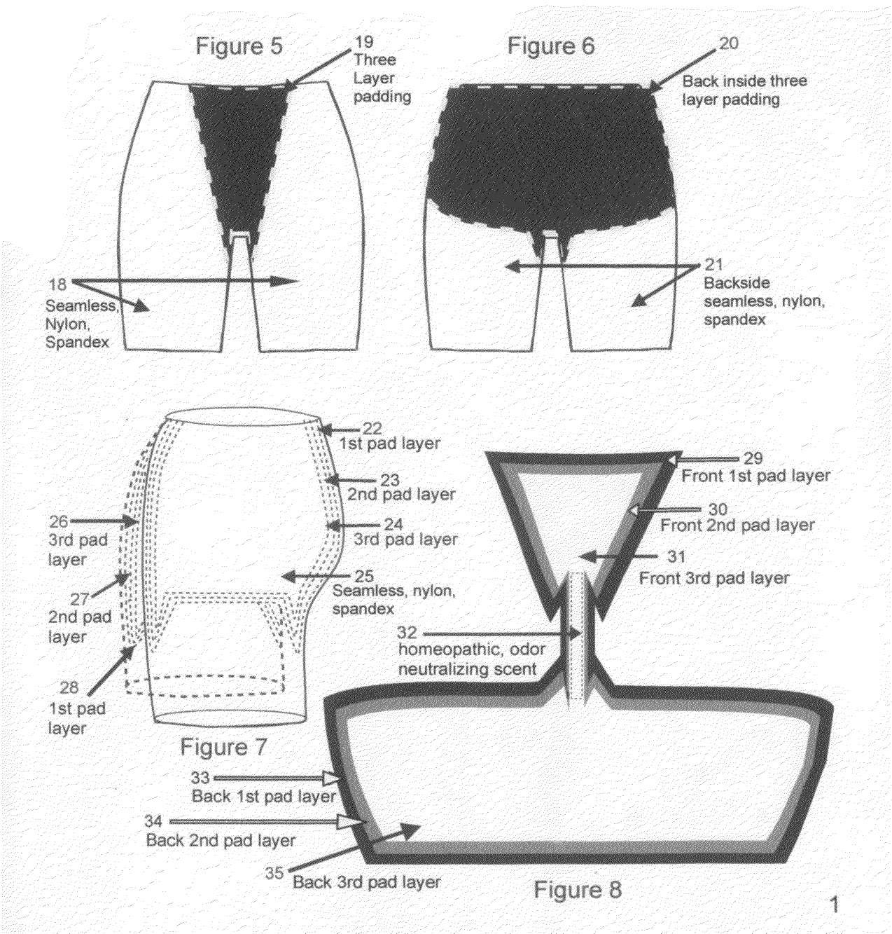

FIG. 5 Front View:
18. Seamless Nylon Spandex
19. Three Layer Padding

FIG. 6 Back View:
20. Back Side in Three layer padding
21. Backside Seamless, Nylon, Spandex

FIG. 7 Side View Layered:
22. First Pad Layer (Back)
23. Second Pad Layer (Back)
24. Third Pad Layer (Back)
25. Seamless, Nylon, Spandex (Back)
26. Third Pad Layer (Front)
27. Second Pad Layer (Front)
28. First Pad Layer (Front)

FIG. 8 Inside View Layered:
29. First Pad Layer (Front)
30. Second Pad Layer (Front)
31. Third Pad Layer (Front)
32. Homeopathic Neutralizing Scent
33. First Pad Layer (Back)
34. Second Pad Layer (Back)

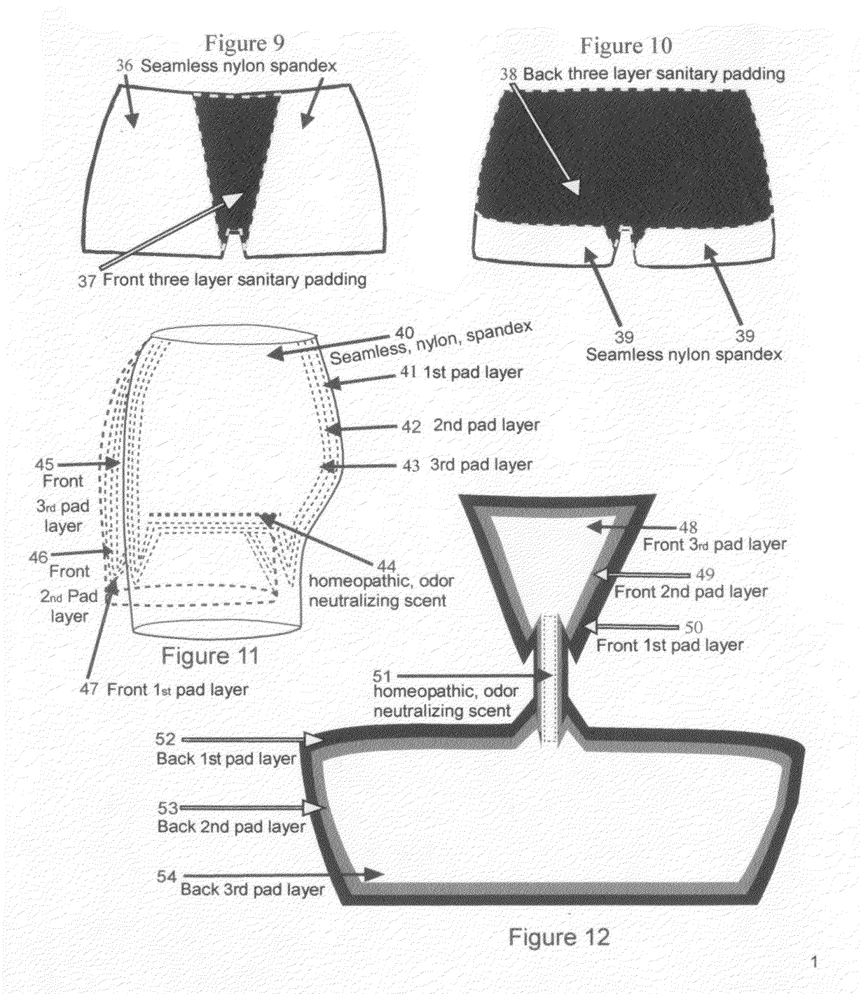

FIG. 9 Front View:
36. Seamless Nylon Spandex (Front)
37. Three Layer Sanitary Padding (Front)

FIG. 10 Back View:
38. Three layer sanitary padding (Back)
39. Seamless Nylon Spandex (Back)

FIG. 11 Side View Layered:
40. Seamless Nylon Spandex
41. First Pad Layer (Back of Shorts)
42. Second Pad Layer (Back of Shorts)
43. Third Pad Layer (Back of Shorts)
44. Homeopathic, Oder Neutralizing Scent
45. Third Pad Layer (Front of Shorts)
46. Second Pad Layer (Front of Shorts)
47. First Pad Layer (Front of Shorts)

FIG. 12 (Inside View):
48. Third Pad Layer (Front)
49. Second Pad Layer (Front)
50. First Pad Layer (Front)
51. Homeopathic, odor Neutralizing Scent
52. First Pad Layer (Back)
53. Second Pad Layer (Back)
54. Third Pad Layer (Back)

1. This product is like no other, a comprehensive designer like, disposable all over coverage, fluid absorbent, anti-leakage, odor neutralizing feminine undergarment that slims, shapes, smoothes, contours the tummy, waist, hips, thighs and rear areas of the body. This product is available in bikini, girl short/ hot pant and thigh length.
